(19) United States
(12) Patent Application Publication
(10) Pub. No.: US 2010/0266517 A1
(43) Pub. Date: Oct. 21, 2010

(54) Two-Coat Cosmetic Compositions

(75) Inventors: Ajay G. Dingley, New Windsor, NY (US); Thi N. Do, West Orange, NJ (US); John R. Glynn, Ridgewood, NJ (US)

Correspondence Address: AVON PRODUCTS, INC., AVON PLACE, SUFFERN, NY, 10901, US

(73) Assignee: Avon Products. Inc., New york, NY (US)

(21) Appl. No.: 12/090,293

(22) PCT Filed: Dec. 28, 2006
(86) PCT No.: PCT/US06/99446

§ 371 Date: Apr. 15, 2008

(51) Int. Cl. A61K 8/87; A61K 8/81; A61Q 17/04; A61Q 15/00; A61Q 1/02; A61Q 3/02

(52) U.S. Cl. 424/59; 424/65; 424/63; 424/61; 8/405

(57) ABSTRACT

Two-coat compositions and methods are provided for forming cosmetic films on a biological surface which are long-lasting, transfer-resistant, water-proof, oil-proof, non-migrating, comfortable to wear, and which are capable of filling in fine lines and wrinkles. The two-coat compositions generally comprise a hydrophilic base coat composition and a top coat composition, and are useful for formulating cosmetics and personal care products.

### CROSS-REFERENCE TO PRIOR APPLICATION

This application claims the benefit of PCT Application Serial No. PCT/US06/499-446 filed Dec. 28, 2006 which in turn claims priority to U.S. provisional application U.S. Ser. No. 60/755,674 filed Dec. 30, 2005.

### FIELD OF INVENTION

The present invention relates generally to compositions and methods for improving the appearance of skin. More particularly, the present invention relates to cosmetic compositions which are uniform, long-lasting, transfer-resistant, non-migrating, and comfortable to wear, and which are capable of filling in fine lines and wrinkles to yield a flawless appearance of the skin.

### BACKGROUND OF THE INVENTION

There has been increasing interest in formulating a variety of cosmetic products that camouflage fine lines and wrinkles by filling them in without excessive buildup, in order to provide an aesthetically appealing, or flawless appearance, that at the same time are transfer-resistant (or non-transferable), long-lasting, and comfortable to wear. To date, the majority of cosmetics have been unable to meet all of these design criteria. Traditional efforts to formulate such cosmetics have focused on the use of silicone resins and volatile silicone oils which have improved properties regarding longer wear, but which have the drawback of leaving an uncomfortable film on the skin or lips.

Currently available cosmetic compositions that are applied to the skin or lips, including, for example, foundations and lipsticks have a tendency to migrate from the surface to which they are applied. Migration refers to the running of the composition, and in particular, of the color, beyond the initial line of where the composition was applied. Migration of such cosmetics, such as when applied to the skin, may lead to an unpleasant appearance around the lips and the eyes, which particularly makes wrinkles and fine lines more prominent. This migration is often mentioned by consumers as being a major defect of, for example, conventional lipsticks, foundations, concealer products and eye make-ups. In addition, these compositions generally are not long-lasting, leading to modifications of the color which generally follow an interaction with the sebum and sweat secreted by the skin, or with saliva on the lips. The resulting smearing or migration leads to an uneven coating and uneven color, requiring the user to reapply the cosmetic frequently.

Generally, cosmetic compositions seeking to have the advantageous properties of being transfer-resistant and long-lasting have employed a single emulsion coat. Typical examples of such cosmetic compositions include the following U.S. patents:

U.S. Pat. No. 5,876,736 provides emulsified, pigmented cosmetic compositions, preferably as oil-in-water emulsions to tighten the skin and reduce the appearance of fine lines. The compositions comprise film-forming agents comprising plant polysaccharides and hydrolyzed casein.

U.S. Pat. No. 5,560,917 discloses emulsified, pigmented compositions that are preferably water-in-oil emulsions for smoothing and protecting skin.

U.S. Pat. No. 4,119,712 provides dry cosmetic compositions comprising a mixture of hydrophobic fumed silica and hydrophilic fumed silica to increase adhesion, color stability, and length of wear.

U.S. Pat. No. 6,083,516 discloses pigmented, cosmetic compositions comprising styrene-ethylene, propylene block copolymers which are not water soluble but are suitable for use in water-based and anhydrous systems.

Some prior art compounds attempt to provide smooth, durable films for cosmetic use by employing two-step formulations involving a base coat and a top coat, such formulations typically employing an emulsion base coat. Such cosmetics are disclosed in, for example, U.S. Pat. No. 6,811,770. However, in practice, the use of emulsion base coats has been found to be undesirable, as it leads to smearing of the top coat and base coat, reducing the appearance of smoothness.

Therefore, despite advances in film-forming methods and compositions, there is currently a need for cosmetic products that camouflage and fill in fine lines and wrinkles without excessive buildup, in order to provide an aesthetically appealing or flawless appearance, and that are also long-lasting, non-transferable, non-migrating, and comfortable for consumers over long periods of wear time. Although many approaches have been used in the art, presently, cosmetic products on the market consistently fail to provide products that satisfy the above-mentioned criteria.

It is therefore an object of the present invention to provide cosmetic compositions that fill in fine lines and wrinkles on skin (including the lips), thereby camouflaging their appearance resulting in a flawless look, and that are long-lasting, non-transferable, non-migrating, and comfortable, which can be applied to biological surfaces.

### SUMMARY OF THE INVENTION

Surprisingly, it has been found that inventive two-coat compositions, which comprise a hydrophilic base coat composition and a top coat composition, form a cosmetic film on a biological surface that exhibits superior properties compared to compositions of the prior art. Specifically, the inventive two-coat composition has the ability to fill in fine lines and wrinkles, yielding a flawless appearance of, for example, the skin. These two-coat compositions of the invention also have the advantage of being transfer-resistant and migration-resistant, and are also uniform in color, long lasting, and comfortable to the wearer.

One aspect of the invention provides methods for forming cosmetic films on a biological surface. The biological surface may be any surface to which cosmetics or personal care products are typically applied, including without limitation skin, lips, keratin fibers, nails and the like. Generally, the method comprises applying a base coat composition to a biological surface to form a base coat. The base coat composition comprises at least one water-soluble film former that is capable of forming a hydrophilic film on the biological surface. The method also comprises applying a top coat composition on the base coat to form a top coat which may be applied over all or part of the base coat. The base coat and top coat form a hydrophobic cosmetic film on the biological surface. The resulting cosmetic film is water-resistant, oil-resistant, migration-resistant, smooth, durable, and capable of filling in fine lines and wrinkles, yielding a flawless appearance of skin.

Another aspect of the invention provides methods for entrapping a functional agent within a cosmetic film on a biological surface. The functional agent may be any agent which is desired to be held in intimate contact with the surface, including without limitation pigments, pharmaceuticals, cosmeceuticals and the like. The method comprises applying a base coat composition to the biological surface to form a base coat, and applying a top coat composition on the base coat to form a top coat. The base coat and the top coat form a hydrophobic cosmetic film on the biological surface, and a functional agent is entrapped within the cosmetic film.

An additional aspect of the invention provides two-coat compositions that comprise a base coat composition having at least one water-soluble film former capable of forming a hydrophilic film on a biological surface, and a top coat composition having at least one film former. In yet another embodiment of the invention, the two-coat composition is a cosmetic or personal care product. Yes it is in this case to allow for the top coat to exhibit dimensional stability.

A further embodiment of the invention relates to two-coat compositions in which the base coat composition of the two-coat composition is not an emulsion.

In another embodiment of the invention, the base coat composition of the two-coat composition further comprises one or more coloring agents.

In another embodiment of the invention, the top coat composition of the two-coat cosmetic composition further comprises one or more coloring agents.

Yet another embodiment relates to a foundation or concealer which utilizes the inventive two-coat composition for concealing, camouflaging, or hiding fine lines or wrinkles on the skin upon application of the foundation or concealer, yielding a flawless appearance of the skin. These and other aspects of the invention will be better understood by reference to the following detailed description and appended claims.

### DETAILED DESCRIPTION OF THE INVENTION

In the following description of the invention and the claims appended hereto, it is to be understood that the terms used have their ordinary and accustomed meanings in the art, unless otherwise specified. All concentration are by weight percent on an active basis unless otherwise indicated.

Generally, the present invention provides two-coat compositions or cosmetic products that may deliver uniform color to the skin, nails, or keratin fibers. The inventive two-coat compositions generally comprise a base coat composition having at least one water-soluble film former capable of forming a hydrophilic film on a biological surface and a top coat composition. Typically, the top coat composition contains at least one film former.

One embodiment of the invention is a two-coat cosmetic composition comprising a base coat composition comprising at least one water-soluble film former capable of forming a hydrophilic film on a biological surface, and a top coat composition comprising a hydrophobic emulsion and preferably including at least one hydrophobic film former. The two-coat composition may further comprise one or more coloring agents.

Another embodiment of the invention is a two-coat cosmetic composition comprising a base coat composition comprising at least one water-soluble film former capable of forming a hydrophilic film on a biological surface, and an anhydrous top coat composition preferably including a hydrophobic film former.

The present invention also provides methods for forming a cosmetic film on a biological surface comprising applying a base coat composition to a biological surface to form a base coat. The base coat composition comprises at least one water-soluble film former that is capable of forming a hydrophilic film on the biological surface. The base coat may be permitted to dry in whole or in part. The method also comprises applying a top coat composition on the base coat to form a top coat which may be applied over all or part of the base coat. The base coat and top coat form a hydrophobic cosmetic film on the biological surface. By ‘cosmetic film’ is meant the composite film of the product obtained by the sequential application of the base coat composition and the top coat composition.

The inventive resulting cosmetic films have the advantage of being durable (i.e., long-lasting), transfer-resistant, oil resistant, water-resistant, non-migrating, and comfortable to the wearer, as well as being able to fill in fine lines and wrinkles where the two-coat compositions are in the form of foundations, concealers, or lip cosmetics.

Without wishing to be bound by any particular theory, it is believed that the benefits of the present invention arise by virtue of limited compatibility between the hydrophilic base coat and the top coat, enabling hydrogen bonding with the OH groups of the base coat, which provides increased adhesion of the top coat to the base coat. The inventive cosmetic films provide heretofore unachievable levels of transfer resistance, migration resistance, smoothness, and comfort in cosmetics, while filling in lines and wrinkles to approach a flawless appearance in the case of a cosmetic foundation, and also for personal care products.

The resulting cosmetic film acts as a moisture barrier, thereby decreasing moisture sensitivity. The two-coat compositions of the invention and the formed cosmetic films of the invention are particularly superior to commercially-available products that utilize an emulsion, or hydrophobic base coat in terms of the above-described performance factors.

Where the product is a two-coat cosmetic foundation or concealer, without wishing to be bound by theory, the base coat and/or top coat fills in fine lines and wrinkles of the skin, and blurs imperfections, adding a first degree of smoothness while the top coat provides further coverage of the base coat, yielding a smooth, even, substantially flawless appearance or finish on the skin. The top coat enhances the smoothness by providing an even coverage of the base coat. “Flawlessness” refers to consumer perception of wrinkle reduction, age spots, imperfections in the skin, and discoloration on the skin. Smoothness is also a factor that contributes to the flawless finish on the skin. By “flawless” is meant skin that appears natural-looking skin, i.e., essentially no caking off of the composite cosmetic film.

### Base Coat Composition

Water-soluble film forming polymers are contemplated to be useful in the base coat composition of some embodiments of the invention.

Water-soluble film formers useful in the practice of the invention can be based on several types of systems, including but not limited to PVP, acrylates, and urethanes; synthetic polymers of the polycondensate type or free-radical type, or ionic type, polymers of natural origin and mixtures thereof or any other film former known within the practice of the cosmetic and pharmaceutical arts which one skilled in the art may determine to be compatible with the compositions of the invention. Specific examples of water-soluble film forming polymers suitable for use in the invention include those disclosed in U.S. Pat. No. 6,939,536 (Chen et al.), the contents of which are hereby incorporated by reference.

More specifically, the water-soluble film former may be chosen from, for example, vinylpyrrolidone/vinyl acetate (PVP/VA) copolymers such as the Luviskol® VA grades (all ranges) from BASF® Corporation and the PVP/VA series from ISP; acrylic fluorinated emulsion film formers including Foraperle® film formers such as Foraperle® 303 D from Elf Atochem; GANEX® copolymers such as butylated PVP; Poly(vinylpyrrolidone/diethylaminoethylmethacrylate) or PVP/Dimethylaminoethylmethacrylate copolymers such as Copolymer 845; Resin ACO-5014 (Imidized IB/MA copolymer); other PVP based polymers and copolymers; alkyl cycloalkylacrylate copolymers (See WO 98/42298, the disclosure of which is hereby incorporated by reference); Mexomere® film formers and other allyl stearate/vinyl acetate copolymers (allyl stearate/VA copolymers); polyolprepolymers such as PPG-12/SMDI copolymer, Poly(oxy-1,2-ethanediyl), α-hydro-ω-hydroxy-polymer with 1,1′-methylene-bis-(4-isocyanatocyclohexane) available from Barnet; Avalure™ AC Polymers (Acrylates Copolymer) and Avalure™ UR polymers (Polyurethane Dispersions), available from BFGoodrich.

Other non-limiting useful water-soluble film formers according to one embodiment of the invention include cellulosic polymers such as, for example, hydroxyethyl cellulose, hydroxypropyl cellulose, carboxymethyl cellulose, hydroxypropyl ethyl cellulose, and ethyl hydroxyethyl cellulose; keratin derivatives such as, for example, keratin hydrolysates and sulfonic keratins; anionic chitins, cationic chitins, amphoteric chitins, nonionic chitins and chitosan derivatives, such as hydroxypropyl chitosan; cellulose derivatives and quaternized derivatives of cellulose; acrylic polymers and acrylic copolymers including, for example, polyacrylates and polymethacrylates; polyvinyl alcohols (PVA) and polyvinylpyrrolidones (PVP); vinyl copolymers such as copolymers of methyl vinyl ether and malic anhydride, and copolymers of vinyl acetate and crotonic acid; polyethylene glycols (PEG); and modified polymers of natural origin including from: gum arabic, guar gum, xanthan derivatives, karaya gum; alginates, carrageenans, glycoaminoglycans, hyaluronic acid, hyaluronic acid derivatives, shellac resin, sandarac gum, dammars, elemis, copals, and deoxyribonucleic acid.

Preferred water-soluble film formers according to one embodiment of the invention include polyvinylpyrrolidones, polyvinyl alcohols, polyurethanes, polyethylene glycols and PVA-PEG copolymers.

In one preferred embodiment of the invention, the base coat composition comprises Kollicoat® Protect which is commercially available from BASF and comprises between about 55% and about 65% alcohol-polyethylene glycol graft copolymers, between about 35% and 45% polyvinyl alcohol, and between about 0.1% and about 0.3% silicon dioxide.

In another preferred embodiment of the invention, the base coat composition comprises Kollicoat® IR, commercially available from BASF, which comprises about 75% polyvinyl alcohol, about 25% polyethylene glycol, and about 0.3% silica.

In further embodiments of the invention, the base coat composition may comprise combinations of various water-soluble film formers, such as, for example, combinations comprising Kollicoat® Protect and Kollicoat® IR.

The concentration of the film former may be determined by one of skill in the art and can vary considerably based on the application. For example, for some cosmetic compositions of the invention, film formers may be used in an amount from 0.1% to 99.0% by weight, relative to the total weight of the composition.

Preferably, film formers in the invention are used in an amount from 1.0% to 40.0% by weight, relative to the total weight of the composition.

More preferably, film formers in the invention are used in an amount from about 3% to 30%, preferably 3% to about 25%, most preferably from about 5% to about 20% by weight, relative to the total weight of the composition.

### Top Coat Composition

Top coat compositions useful in the cosmetic compositions of the invention comprise at least one film forming polymer. The top coat composition is capable of forming a film that is hydrophobic. Useful top coat compositions in some embodiments of the invention may comprise a cosmetically acceptable emulsion. However, anhydrous top coat compositions are preferred. In some embodiments of the invention, coloring agents are dispersed in the top coat emulsion. In one embodiment of an anhydrous top coat composition, the top coat contains polyvinyl stearyl ether sold as Giovarez 1800 (MW 3000-4000) by Phoenix Chemical, a 100% active, water insoluble, waxy, non-ionic resin that forms a continuous, glossy, somewhat soft film when cast from a molten state.

An emulsion is a mixture of two immiscible liquids in which droplets of one liquid (the discontinuous or internal phase) are dispersed throughout the other liquid (the continuous or external phase). Emulsions may behave and appear as substantially homogenous fluids. The products of the present invention comprise a cosmetically acceptable vehicle in the form of an emulsion, including but not limited to, water-in-oil, oil-in-oil, polyol-in-oil emulsions. A water-in-oil emulsion is a mixture where water droplets form the discontinuous phase which is dispersed in a continuous oily phase. Conversely, an oil-in-water emulsion is a mixture where water-insoluble droplets are dispersed in a continuous aqueous phase

Other emulsions such as water-in-silicone, silicone-in-water, and silicone-in-oil emulsions are also contemplated to be useful in the practice of the invention.

Multiple phase emulsions such as water-in-oil-in-water emulsions are also contemplated to be useful according to the invention. These emulsions comprise droplets of an emulsion dispersed in another emulsion. For example, a water-in-oil-in-water emulsion comprises a continuous water phase containing dispersed oil droplets which is itself dispersed in a continuous oil phase. Double emulsions and multiple emulsions are also contemplated to be useful in the practice of the invention.

Where the top coat composition of the two-coat composition comprises an emulsion, it is preferred that the top coat composition of the two-coat composition comprises a water-in-oil emulsion. The aqueous phase preferably comprises demineralized water. The other water soluble or water dispersible components of the composition would also be incorporated into the aqueous phase. The oil phase may be any oil conventionally used in cosmetic formulations. Exemplary oils include, but are not limited to fatty alcohols, hydrocarbon oils, natural or synthetic triglycerides, waxes and wax-like materials, esters of long-chain acids and alcohols, silicone oils, fatty acid esters, lanolin and lanolin derivatives, and combinations thereof. Specific examples of oils suitable for use in the invention include those disclosed in U.S. Pat. No. 6,521,217 (Luther et al.) and 5,876,736 (Cohen et al.), the contents of which are hereby incorporated by reference.

Any emulsifier may be used with the present invention. Suitable emulsifiers include ethoxylated esters of a natural oil derivative such as polyethoxylated esters of hydrogenated castor oil, silicone oil emulsifiers such as silicone polyols, soaps such as, for example, fatty acid soaps and ethoxylated fatty acid soaps, ethoxylated fatty alcohols, optionally ethoxylated sorbitan esters, ethoxylated fatty acids, and ethoxylated glycerides. Specific examples of suitable emulsifiers are disclosed in U.S. Pat. No. 6,290,936 (Ross), the contents of which are hereby incorporated by reference.

Where the invention comprises an emulsifier, it is preferred that the emulsifiers are solid at room temperature, including, but not limited to emulsifiers such as behenyl alcohol, Steareth-2, PEG-40 stearate, and glyceryl stearate. The emulsions may further comprise any of the thickeners well known in the art. A preferred thickener for use in the emulsions of the present invention is the carbomer available as Carbopol® 940 (Noveon, Ohio).

When formulated as an oil-in-water emulsion, the cosmetically acceptable vehicle preferably comprises 5 to 50% of an oil phase and 30 to 90% of water, each by weight based on the total weight of the emulsion, with the remainder optionally comprising an emulsifier.

Film formers that are contemplated to be useful in the top coat composition of the invention are preferably hydrophobic, and can be silicones, fatty acid esters, aliphatic and/or oligomeric, and mixtures thereof. The film formers include, but are not limited to, polyethylene film formers, silicone acrylate, and hydrogenated cyclopentadiene.

In one preferred embodiment of the invention, the top coat composition comprises polyvinyl stearyl ether, which is commercially available from Phoenix Chemical, Inc. as Giovarez 1800.

### Properties

In a further embodiment of the invention, various elements may be used in either the top coat composition or the base coat composition.

For example, the film-forming polymers used according to the invention have a glass transition temperature (Tg) in a range which causes a soft and elastic film to be produced. Glass transition temperatures are the point at which the polymer or fragment thereof moves from a solid brittle state into a rubbery liquid state. As will be understood by one skilled in the art, the Tg of various polymers may be determined through testing and by referring to the glass transition points which are described in commonly known and used references (see, Polymer Handbook, Eds. J. Brandrup, et al., 2 Volumes Set, Fourth Edition, John Wiley and Sons, Inc., June 2003; Introduction to Polymer Science and Technology, Eds. H. S. Kaufman and J. J. Falcetta, John Wiley and Sons, Inc., 1977) in order to understand this concept and to identify particular combinations of polymers which would be useful in the invention.

The Tg of the materials useful in both the base coat composition and the top coat composition is typically between about −30° C. and about 175° C., and preferably between about 0° C. and about 75° C.

Preferably, the molecular weight of the materials useful in both the base coat composition and the top coat composition is between about 10 Kdaltons and about 1200 Kdaltons.

The degree of adhesion between the two coats is greater when the surface energies of the two coats are compatible. It is preferred that the surface energies of both the base coat and the top coat are between about 18 and 75 dynes/cm as measured by a peel adhesion test (described in Example 5).

Typical refractive indices of both the base coat composition and the top coat composition are between about 1.3 and about 2.8. It is preferred that the refractive indices of both the base coat composition and the top coat composition are between about 1.4 and about 2.1.

### Other Materials

Other ingredients may be present in either the base coat composition, the top coat composition, or in both coat compositions. These ingredients include, but not limited to functional agents such as pigments, lakes, dyes, opacifiers, pearling agents, insect repellents, UV absorbers, UV blockers, antiperspirants, anti-acne agents, moisturizers, conditioners, and tooth whiteners, as well as coloring agents, fillers, preservatives, soft focus materials, tightening agents, solvents, carriers, vehicles, and the like.

In some embodiments of the invention, the base coat composition of the cosmetic product further comprises one or more coloring agents.

In some embodiments of the invention, the top coat composition of the cosmetic product further comprises one or more coloring agents.

In further embodiments of the invention, both the base coat composition and the top coat composition of the cosmetic product do not comprise coloring agents.

Coloring agents may be added where the product is a cosmetic or personal care product, including, for example, lipstick, lip gloss, nail enamel, foundation, and the like. One or more coloring agents may be added to the base coat, the top coat, or to both coats. It is within the skill in the art to choose coloring agents and combinations of coloring agents to produce a desired color. Suitable coloring agents, including pigments, lakes, and dyes, are well known in the art and are disclosed in the C.T.F.A. International Cosmetic Ingredient Dictionary and Handbook, Tenth Edition, 2004, the contents or which are hereby incorporated by reference. Organic pigments include, for example, FD&C dyes, D&C dyes, including D&C Red, Nos. 2, 5, 6, 7, 10, 11, 12, 13, 30 and 34, D&C Yellow No. 5, Blue No. 1, Violet No. 2. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, y-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Other suitable coloring agents include ultramarine blue (i.e., sodium aluminum silicate containing sulfur), Prussian blue, manganese violet, talc, mica, sericite, carbon black, magnesium carbonate, calcium carbonate, magnesium silicate, aluminum magnesium silicate, silica, titanated mica, iron oxide titanated mica, bismuth oxychloride, and the like. The coloring agents may be surface modified, with, for example, fluoropolymers, to adjust one or more characteristics of the coloring agent as described in, for example, U.S. Pat. Nos. 6,471,950, 5,482,547, and 4,832,944, the contents of which are hereby incorporated by reference. Fluoropolymers may be incorporated into the present invention as a coating on pigment particles that at least partially covers the surface of the pigment particles. Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference.

The compositions of the invention may optionally comprise other active and inactive ingredients, including, but not limited to, excipients, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, coloring agents, staining agents, depigmenting agents, hypopigmenting agents, preservatives, stabilizers, pharmaceutical agents, photostabilizing agents, and mixtures thereof. In addition to the foregoing, the personal care products of the invention may contain any other compound for the treatment of skin disorders.

Fillers useful in the present invention may be mineral or organic, and lamellar, spherical or oblong. The filler may be chosen from talc, mica, silica, kaolin, polyamide powders such as Nylon® (Orgasol® from Atochem) powder, poly-β-alanine powder, polyethylene powder, acrylic polymer powder, polymethyl methacrylate (PMMA) powder, for instance the product sold or made by Wacker under the reference Covabead LH-85 (particle size 10-12 μm) or acrylic acid copolymer powder (Polytrap® from Dow Corning), polytetrafluoroethylene (Teflon®) powders, lauroyllysine, boron nitride, starch, hollow polymer microspheres such as those of polyvinylidene chloride/acrylonitrile, for instance Expancel® (Nobel Industrie), hollow polymer microspheres (Tospearl® from Toshiba, for example), precipitated calcium carbonate, magnesium carbonate and hydrocarbonate, Celluflow TA-25 (from Collaborative), hydroxyapatite, hollow silica microspheres (Silica Beads® from Maprecos), glass or ceramic microcapsules and polyester particles. The fillers may be surface-treated, e.g., to make them lipophilic.

The two-coat compositions of the invention may optionally comprise materials that prevent the appearance of fine lines and wrinkles on the skin by blurring their ability to be observed, including soft focus materials, such as optical diffusers.

Non-limiting examples of soft focus materials suitable for the present invention include calcium aluminum borosilicate, PMMA, polyethylene, polystyrene, methyl methacrylate, titanium dioxide, boron nitride, sericite, mica talc, silicon hybrid powders such as Gransil USQ and ESPQ, Velvesil 125, and silica, or mixtures thereof.

Non-limiting examples of preservatives suitable for the present invention include imidazonidyl urea, methylparaben, propylparaben, butylparaben, ethylparaben, methylisothiazolinone, and phenoxyethanol, or mixtures thereof.

The two-coat cosmetic compositions of some embodiments of the invention may also comprise a cosmetically acceptable carrier or vehicle acceptable for topical application to the skin, keratin fibers, or nails. The polymers of the invention may be soluble in the carrier, or they may be suspended as a colloidal dispersion in the vehicle.

Examples of such vehicles include, but are not limited to, water and aqueous systems; glycerin; various hydrophilic solvents including alcohols such as ethanol, methanol, propanol and other alcohols; or any combinations thereof. Hydrocarbon solvents useful in the practice of the invention, in some embodiments, may be chosen from mineral oils, mineral solvents, mineral spirits, petrolatum, waxes, synthetic hydrocarbons, animal oils, vegetable oils, and mixtures of various hydrocarbons. In other embodiments, typical cosmetic esters can be used, including isopropyl palmitate and isopropyl myristate In some embodiments, isododecane or a light paraffinic solvent are used. In other embodiments, a non-hydrocarbon solvent such as amyl acetate, butyl acetate, isobutyl acetate, ethyl acetate, propyl acetate or isopropyl acetate are used.

In addition, the vehicle of the compositions according to the present invention can be in the form of a suspension, gel, or solution, formulation. Other suitable topical carriers include an anhydrous liquid solvent such as oil and mono- or polyhydric alcohols; aqueous-based single phase liquid solvent (e.g. hydro-alcoholic solvent system); anhydrous solid and semisolid (such as a gel and a stick); and aqueous based gel and mousse system.

The product form of the present invention may be, for example, an aerosol, cream, emulsion, gel, liquid, lotion, mousse, pomade, powder, solid, spray, or stick.

The two-coat compositions of the invention are contemplated to be useful in any number of commercial products, including but not limited to over-the-counter drugs and pharmaceuticals, artificial tanning products, sporting camouflage, foot-care products, fragrance-emitting products, analgesics, insect repellents, poison ivy products including poison ivy blocks, and decorative cosmetics including various make-up products, pressed powder, moisturizers, eye liners, body art, lipsticks, lip gloss, lip balms, lip liners, eye shadows, eye creams, nail care products, and, preferably, facial products such as foundations and concealers.

### Two-Coat Compositions

A non-limiting example of the two-coat composition is a cosmetic foundation comprising a base coat composition and a top coat composition. The base coat composition comprises at least one water-soluble film forming polymer, and the top coat comprises at least one film former. Other materials may also be added to the base coat composition, to the top coat composition, or to both coat compositions for the purposes of, for example, coloring, comfort, filling in fine lines and wrinkles, creating a smooth, flawless appearance, and the like. The base coat composition, the top coat composition, or both coat compositions may contribute to the filling in of fine lines and wrinkles, and may provide a smooth, flawless appearance. Additional, non-limiting examples of other materials that may be added to either the base coat composition, the top coat composition, or both coat compositions, include coloring agents, fillers, soft focus materials, preservatives, tightening agents, solvents, vehicles, and carriers, and combinations thereof.

In a preferred embodiment, the two-coat cosmetic or personal care product comprises a base coat of Kollicoat® Protect, Kollicoat® IR, Labrasol, fillers, and water, and the top coat comprises Giovarez 1800, isododecane, pigments, and soft focus materials (see Example 1).

Preferred fillers in the present invention include, but are not limited to talc, mica, silica microspheres, polyurethane beads, and nylon-12.

A preferred soft focus material in some embodiments of the invention is cyclopentasiloxane and C30-45 alkyl dimethicone/polycyclohexene oxide crosspolymer, commercially available from GE as Velvesil 125.

Optionally, the two-coat composition may comprise a tightening agent such as sodium polystyrene sulfonate, albumin, and sodium silicate.

### Method of Manufacture

For manufacture of the base coat composition, the “triple L” blade is used for mixing the polymers into water at the desired concentration under low speed in order to minimize aeration and foaming of the mixtures. The mixing takes place in a beaker at 60° C. until a homogeneous/uniform result is achieved. Moreover, Labrasol (from Gattefosse) is used as a processing aid, since it functions as a defoaming/anti-foaming product. At this point, fillers or other materials are added.

For manufacture of the top coat composition, the film forming polymer is melted with solvent until a desired concentration is reached. At this point, pigments and other soft-focus materials are added.

The invention also provides kits comprising a base coat composition, a top coat composition, and instructions for application of the compositions to a biological surface, such as, for example, skin. The base coat composition comprises as least one water-soluble film former. Optionally, the kit may comprise applicators for applying the base coat composition and the top coat composition.

### Methods of Use

Another embodiment of the invention provides methods for forming a cosmetic film on a biological surface. Generally, the method comprises applying a base coat composition to a biological surface to form a base coat. The base coat composition comprises at least one water-soluble film former that is capable of forming a hydrophilic film on the biological surface. The base coat is permitted to dry either in part or in whole. The method also comprises applying a top coat composition on the base coat to form a top coat which may be applied over all or part of the base coat. The top coat most preferably comprises at least one film former to enhance wear. The base coat and top coat form a hydrophobic film on the biological surface. The resulting cosmetic film is water-resistant, oil-resistant, migration-resistant, smooth, durable, and capable of filling in fine lines and wrinkles, yielding a flawless appearance of skin.

Another aspect of the invention provides methods for entrapping a functional agent within a cosmetic film on a biological surface. The functional agent may be any agent which is desired to be held in intimate contact with the surface, including without limitation pigments, pharmaceuticals, cosmeceuticals and the like. The method comprises applying a base coat composition to the biological surface, forming a base coat, and applying a top coat composition on the base coat, forming a top coat. The base coat and the top coat form a hydrophobic cosmetic film on the biological surface, and a functional agent is entrapped within the cosmetic film.

### Example 1

Table 1 provides illustrative formulations of cosmetic foundation products comprising a base coat and a top coat.

TABLE 1

| Base Coat | Top Coat |
|---|---|
| Water: q.s. 100% | Giovarez 1800: 5-50% |
| Kollicoat ® Protect: 2-30% | Isododecane: 30-60% |
| Kollicoat ® IR: 1-25% | Pigments: 5-40% |
| Fillers: 5-20% | Soft focus materials: 1-15% |
| Labrasol: 0.25-0.75% | |

### Example 2

Base coat Compositions A and B were prepared along with top coat Composition C, as set forth in Tables 2 and 3 below.

TABLE 2

| | Composition A | Composition B |
|---|---|---|
| Ingredients | (wt. %) | (wt. %) |
| Water | q.s. 100% | q.s. 100% |
| Bentone ¹ | — | 1 |
| Kollicoat Protect ² | 8 | 5 |
| PVA 540 ³ | 1 | 1 |
| Kaolin | 5 | 0.5 |
| Magnesium Aluminum | 2 | 2.5 |
| Silicate | | |

¹ A powdered 50:50 blend of hectorite and hydroxyethylcellulose availble from Elementis Specialties Inc.
² Kollicoat Protect comprises between about 55% and about 65% alcohol-polyethylene glycol graft copolymers, between about 35% and 45% polyvinyl alcohol, and between about 0.1% and about 0.3% silicon dioxide, and is available from BASF.
³ PVA 540 is Celvol 540 is from Celanese

TABLE 3

| Ingredients | Composition C (wt. %) |
|---|---|
| Isododecane | q.s. 100% |
| Giovarez 1800 ¹ | 5 |
| Acrylate Co./Isododecane ² | 5 |
| Wax ⁶ | 5 |
| Nylon ³ | 5 |
| Yellow Iron Oxide | 1.0 |
| TiO₂ | 6.6 |
| Red Iron Oxide | 0.4 |
| Brown Iron Oxide | 0.6 |
| Lucentite SAN ⁴ | 5 |
| BPD-800 ⁵ | 2 |
| Crodaperse ⁷ | 2 |

¹ Polyvinyl stearyl ether available from Phoenix Chemical; 100% active.
² A 50:50 liquid blend of Acrylates Copolymer in Isododecane available from Phoenix Chemical as Giovarez AC-5099 ML.
³ Nylon 12 sold as Orgasol 2002.
⁴ Lithium Magnesium Silicate Kobo Products Inc.
⁵ A powdered blend of polyurethane (95-97%) and Silica (3-5%) having an average particle size of 5 to 7 microns available from Kobo Products Inc.
⁶ Dow 2501 Cosmetic Wax, a polymer of dimethylsiloxane with polyoxyethylene and/or polyoxypropylene side chains and containing 0.05% BHT.
⁷ Caprylic/Capric Triglyceride, Di-PPG-3 Myristyl Ether Adipate and Sorbitan Isostearate blend from Croda.

The ability of the base coat Compositions A and B and of the top coat Composition C to form a smooth film were examined and compared to the commercial product Covergirl© Outlast© (trademarks of The Procter and Gamble Company) foundation, which consists of a base coat and top coat. The testing protocol is described below.

### Test Method For Evaluating “Smoothness”

This method can be used to determine “smoothness” after a cosmetic product is applied to a “non-skin” surface that emulates skin.

Roughness parameters of a substrate were measured pre- and post-application of the product. Base coat compositions and top coat compositions were applied to circular polyurethane discs (“non-skin”) obtained from _US cosmetics Corporation, Dayville, Conn. and readings were taken pre- and post-application of product using the Visioscan 98 and the SELS (Surface Evaluation of Living Skin) 2000 software. On average, women use 0.6 g of foundation for their face (approx. area of 200 cm²). The disc to which the product was applied has an area of 20 cm². Certain areas on the disc were marked (6×8 mm). The product (0.06 g) was deposited on the disc and a height-adjustable drawdown bar was used to obtain a uniform film of 15 mils in wet thickness. Prior to taking any images, the product was allowed to dry for 15 minutes. The image of the marked area was then taken by a built-in CCD-camera. The connection of the Visioscan VC 98 to the PC was done via an image digitalization unit which configures the image in 256 gray level pixel by pixel, where 0 is black and 255 is white. The SELS software calculated surface and texture roughness parameters. The differences in roughness levels were recorded and the percentage improvement was calculated using the pre-application values as a baseline. Positive values indicate an increase in smoothness, and negative values indicate an increase in roughness. This procedure was performed with both the base coat compositions and the top coat compositions.

TABLE 4

| Product | Percentage Improvement |
|---|---|
| Covergir ©l Outlast © base | −29.8 |
| coat | |
| Covergirl © Outlast © top | +26.3 |
| coat | |
| Composition A base coat | +30.6 |
| Composition C top coat | +32.6 |

The results indicated that the Compositions A and C exhibit superior “smoothness” readings compared to the Covergirl base coat and to coat compositions, respectively. Both the base coat composition and the top coat composition of Example 2 exhibited greater smoothness percentage improvement than did the Covergirl control products.

### Example 3

The ability of the top coat composition described in Example 2 to form a smooth film when applied to their respective base coat compositions was evaluated using PRIMOS (Phaseshift Rapid In Vivo Measurement of Skin) available form GFMesstechnik GmbH.

The optical method used for measuring is the digital stripe projection technique, based on digital micro mirror projectors. Stripes with sinusoidal intensity of brightness are projected onto the surface of the measuring object and the projection, (the stripes changed by the height profile of the measuring object) is recorded at a defined triangulation angle by a CCD camera. The topography of the measuring object is calculated from the stripes' position and the grey value of all registered individual image points. Depending on the measurement task, different stripe projection procedures can be used. The 3D measurement effect is achieved by the fact that minute elevation differences on the surface deflect the parallel projection stripes and that these deflections constitute a qualitative and quantitative measure of the skin profile.

0.06 g of top coat was applied to the 20 cm² substrates as described in Example 2 to which the base coat had been. Images were taken of the marked area before and after application of foundation. Smoothness is determined using this method by ascertaining the smoothness depth R4, which is the area of the void space in the surface of the film between a line at the highest point on the surface of the film and a line that is the mean distance between the high point and the low point of the surface. Percentage improvement was calculated using the pre-application values as a baseline. Positive values indicate an increase in smoothness, and negative values indicate an increase in roughness (decrease in smoothness).

TABLE 5

| | Percentage |
|---|---|
| | Improvement in |
| Product | Smoothness |
| Covergirl © Outlast © Base | +30.6% |
| coat + Top coat | |
| Composition A Base coat + | +80.5% |
| Composition C Top coat | |
| Composition B Base coat + | +84.6% |
| Composition C Top coat | |

This data shows that the two-part compositions of the present invention provided superior smoothness to the synthetic skin substitute than a leading foundation brand.

### Example 4

### Non-Transfer Test

This test predicts the ability of a cosmetic film to resist color transfer to objects contacting the skin. The following procedure was used to perform the non-transfer test on the cosmetic film obtained by the products identified in table 5:
(1) Prepare a 3 by 4 inch sheet of collagen sausage casing by hydrating it in a 90% relative humidity chamber for at least two hours.
(2) Remove the collagen sheet to ambient conditions and immediately lay down on a glass slide, while taping the 4 corners ensuring the exposed surface of the collagen to be flat and free of wrinkles.
(3) Allow the collagen-wrapped slide to equilibrate at ambient conditions for 24 hours.
(4) Draw down thin, uniform films of foundation on the collagen. The film area should be at least 2 by 2 inches.
(5) Allow the cosmetic samples on both the collagen to sit at ambient conditions for 24 hours.
(6) Cut two discs from a clean, white Styrofoam dinner plate using a 1.5 inch diameter circular punch. The surface and edges of each disk should be smooth and even.
(7) Firmly attach using double-sided adhesive tape the discs from step (6) to bottom surface of a 2 kg weight.
(8) Set the weight on top of cosmetic sample applied to the collagen surface so that the disc is in contact with the film. One of the disc is placed onto a drop of water, and the second disc is placed onto a drop of oil.
(9) Grasping the top of the 2 kg weight, carefully rotate the disc through 360 degree while maintaining the 2 kg force on the film. Do not lift or press the weight into the film during the rotating motion to the weight. The entire 360 degree rotation should be completed within a time interval between 3 and 5 seconds.
(10) Lift the weight straight up off the film surface. Carefully remove the disc from the weight avoiding damage to the disc.

The disc was subjectively evaluated with regard to the amount of composition retained thereon. Data obtained from this test demonstrated better non-transfer imparted by the base coat Compositions A and B and by top coat Composition C as compared to the Covergirl base coat and top coat formulations. No. Amount of material transferred onto the disc is estimated subjectively.

### Example 5

### Peel Adhesion Test

Peel tests follow ASTM D 3330: Peel Adhesion Of Pressure Sensitive Tape. The test process followed was identical to ASTM with regard to specified equipment, peel speed, and measurement specifications. However, the test specimen preparation and materials are necessarily different from ASTM. Accordingly, a nylon substrate is used instead of steel; the product is cast on substrate instead of tape applied to substrate, and a breathable nylon fabric backing is used in lieu of a polyester film backing. The peel test provides very repeatable differentiation between the adhesive properties of the foundation to the substrate by virtue of the nylon fabric being peeled off. This test used the Peel Tester Model TA-96 for 180 degree Peel Testing and utilizes a TA.XT2 Texture Analyzer, along with the Texture Expert for Windows Software provided by Stable Micro Systems, LTD., Surrey, UK.

TABLE 6

| | Average Force (g) to Remove Film |
|---|---|
| Product | from Substrate |
| Covergirl base coat and top coat | 0.004 |
| Composition A base coat and | 3.802 |
| Composition C top coat | |
| Composition B base coat and | 4.865 |
| Composition C top coat | |

The data shows that the two-part compositions of the present invention provided superior resistance to delamination from the synthetic skin substitute than a leading foundation brand.

The invention having been described by the foregoing description of the preferred embodiments, it will be understood that the skilled artisan may make modifications and variations of these embodiments without departing from the spirit or scope of the invention as set forth in the following claims.

1. A method for forming a cosmetic film on a biological surface comprising:
applying a base coat composition to said biological surface to form a base coat, said base coat comprising at least one water-soluble film former capable of forming a hydrophilic film on said biological surface and applying a top coat composition on said base coat to form a top coat;
wherein said base coat and said top coat form a hydrophobic cosmetic film on said biological surface.

2. The method of claim 1 wherein said water-soluble film former is a polymer selected from the group consisting of polyvinylpyrrolidones, polyvinyl alcohols, polyurethanes, polyethylene glycols, PVA-PEG copolymers, and combinations thereof.

3. The method of claim 1 wherein said top coat comprises a film former.

4. The method of claim 1 wherein said top coat adheres to said base coat.

5. The method of claim 1 wherein the cosmetic film is water-resistant, transfer-resistant, oil-resistant, migration-resistant, smooth, or durable.

6. The method of claim 1 wherein the cosmetic film further comprises a functional agent.

7. The method of claim 6 wherein said functional agent is selected from the group consisting of pigments, lakes, dyes, opacifiers, pearling agents, insect repellants, UV absorbers, UV blockers, antiperspirants, anti-acne agents, moisturizers, conditioners, and tooth whiteners.

8. A method for entrapping a functional agent within a cosmetic film on a biological surface comprising:
applying a base coat composition to said biological surface forming a base coat; and
applying a top coat composition on said base coat forming a top coat, wherein said base coat and said top coat form a hydrophobic cosmetic film on said biological surface, and wherein a functional agent is entrapped within said cosmetic film.

9. The method of claim 8 wherein said functional agent is selected from the group consisting of pigments, lakes, dyes, opacifiers, pearling agents, insect repellants, UV absorbers, UV blockers, antiperspirants, anti-acne agents, moisturizers, conditioners, and tooth whiteners.

10. A two-coat composition comprising:
(i) a base coat composition having at least one water-soluble film former capable of forming a hydrophilic film on a biological surface; and
(ii) a top coat composition having at least one film former.

11. The two-coat composition of claim 10 wherein the top coat adheres to the base coat, forming a cosmetic film.

12. The two-coat composition of claim 11 wherein the cosmetic film is water-resistant, transfer-resistant, oil-resistant, migration-resistant, smooth, or durable.

13. The composition of claim 10 wherein the base coat further comprises one or more coloring agents.

14. The composition of claim 10 wherein the top coat further comprises one or more coloring agents.

15. The two-coat composition of claim 10 wherein said two-coat composition is a cosmetic or personal care product.

16. A kit comprising:
a base coat composition comprising at least one water-soluble film former, a top coat composition, and instructions for application of said base coat composition and said top coat composition to a biological surface.

17. The kit of claim 16 further comprising one or more applicators.
